(19) United States
(12) Patent Application Publication
(10) Pub. No.: US 2007/0149552 A1
(43) Pub. Date: Jun. 28, 2007

(54) Lyophilized compositions of a triazolopyrimidine compound

(75) Inventors: M. Sherry Ku, Thiells, NY (US); W. James Huang, Hillsborough, NJ (US)

Correspondence Address: WilmerHale/Wyeth, 60 STATE STREET, BOSTON, MA, 02109, US

(73) Assignee: Wyeth, Madison, NJ (US)

(21) Appl. No.: 11/639,642

(22) Filed: Dec. 15, 2006

Related U.S. Application Data
(60) Provisional application No. 60/751,131, filed on Dec. 16, 2005.

(51) Int. Cl. A61K 31/519

(52) U.S. Cl. 514/259.31

(57) ABSTRACT

The present invention relates to lyophilized compositions of a triazolopyrimidine compound, or a hydrate thereof, or a pharmaceutically acceptable salt of Compound I or hydrate thereof; solutions useful in preparing said lyophilized compositions; methods for preparing such compositions; methods of reconstituting the same; kits containing such compositions; and uses of the compositions for the treatment of cancer.

### CROSS REFERENCE TO RELATED APPLICATIONS

This application claims the benefit of priority under 35 U.S.C. §11 9(e) to U.S. Patent Application Ser. No. 60/751,131 filed on Dec. 16, 2005 and is hereby incorporated by reference in its entirety.

### FIELD OF THE INVENTION

The present invention relates to lyophilized compositions of a triazolopyrimidine compound or a pharmaceutically acceptable salt thereof, which is useful as an anti-cancer agent.

### BACKGROUND OF THE INVENTION

A triazolopyrimidine compound of formula (I) (“Compound I”) or a pharmaceutically acceptable salt thereof is disclosed by Zhang et al. in US 2005/0090508, the disclosure of which is incorporated herein by reference in its entirety. Compound I has the following structure: 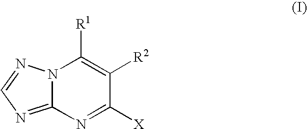
wherein:
R¹ is 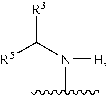
   or (C₆-C₈) cycloalkyl optionally substituted with R⁸;
R² is a moiety of the group 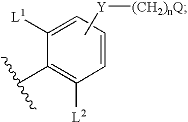
n is an integer of 2, 3, or 4;
X is F, Cl or Br;
Y is O, S, CH₂ or NR⁴;
Q is selected from —NR⁶R⁷ and —OH;
L¹ and L² are each independently H, F, Cl, Br, or CF₃;
R³ is CF₃ or C₂F₅;
R⁴ and R⁵ are each independently H or (C₁-C₃) alkyl;
R⁶ and R⁷ are each independently H or (C₁-C₃) alkyl; or R⁶ and R⁷ may be optionally taken together with the nitrogen atom to which each is attached to form a 4 to 6 membered saturated heterocyclic ring containing 1-2 nitrogen atoms, 0-1 oxygen atoms or 0-1 sulfur atoms, and said 4 to 6 membered saturated heterocyclic ring may be optionally substituted with one or more R⁸; and
R⁸is (C₁-C₃) alkyl.

The triazolopyrimidine compounds of formula (I) bind at the vinca site of β-tubulin, yet they have many properties that are similar to taxanes and distinct from vinca-site agents. In particular, these compounds enhance the polymerization of microtubule-associated protein (MAP)-rich tubulin in the presence of GTP at low compound:tubulin molar ratios, in a manner similar to paclitaxel and docetaxel. The triazolopyrimidine compounds also induce polymerization of highly purified tubulin in the absence of GTP under suitable experimental conditions, an activity that is a hallmark of taxanes. These compounds are potently cytotoxic for many human cancer cell lines in culture, including lines that overexpress the membrane transporters MDR (P-glycoprotein), MRP, and MXR, thus making them active against cell lines that are resistant to paclitaxel and vincristine. In particular, representative examples of this class of triazolopyrimidine compounds have high water solubility and can be formulated in aqueous solution. Representative examples of the triazolopyrimidine compounds are active as anti-tumor agents in athymic mice bearing human tumor xenografts of lung and colon carcinoma, melanoma, and glioblastoma, when dosed either intravenously or orally.

Specifically, a compound of formula (I) having the structure of (Ia) (“Compound Ia”) has been shown to have broad antitumor activity in in-vivo xenograft models of human non-small cell lung cancer (NSCLC), colon cancer, breast cancer, melanoma, and glioblastoma, including models which are resistant to taxanes or other microtubule-active compounds. Compound Ia is 5-chloro-6-{2,6-difluoro-4-[3-(methylamino)propoxy]phenyl}-N-[(1S)-2,2,2-trifluoro-1-methylethyl][1,2,4]triazolo[1,5-a]pyrimidin-7-amine and has the following structure:

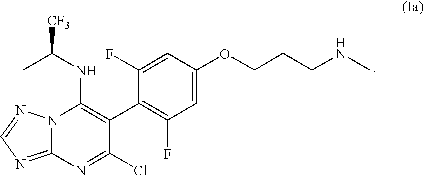

The physical and chemical properties of Compound I result in challenges to the successful formulations of oral and liquid dosage forms due to several mechanisms. For example, Compound I may undergo dimerization and form adducts with acids present in the composition. As a specific example, Compound la undergoes dimerization, as shown in Scheme 1 (the resulting product is hereinafter referred as “Dimer”).

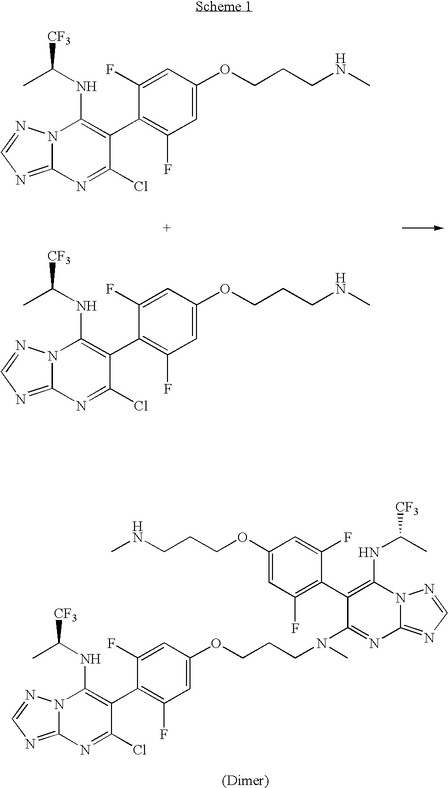

In addition, Compound I may react with carboxylic acid to form an adduct. For example, an amide adduct of Compound Ia is formed by a combination of Compound Ia and succinic acid with the loss of a water molecule as shown below (the product is hereinafter referred as “Adduct”).

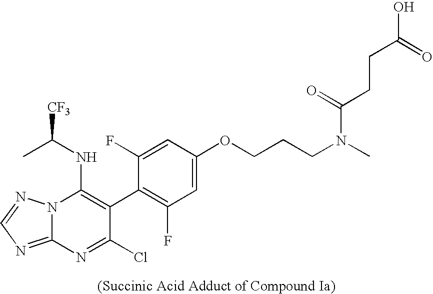

The succinate dihydrate salt of Compound Ia has been found to have high degree of crystallinity, reasonable solubility, and stability and has the following structure as shown below:

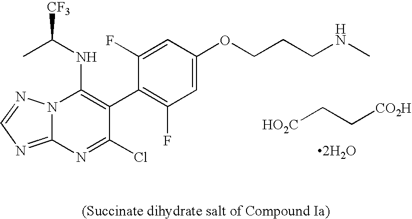

It is a crystalline white to off-white powder with a plate-like crystal habit and is a stable dihydrate in the relative humidity range of 5 to 100%, containing stoichiometric (5.83%) two moles of water. The preferred salt of Compound Ia is the succinate dihydrate salt.

### SUMMARY OF THE INVENTION

The present invention provides lyophilized compositions of Compound I, or a hydrate thereof, or a pharmaceutically acceptable salt of Compound I or hydrate thereof, which overcome the undesirable physical chemical properties of certain triazolopyrimidine compounds. The resulting new compositions provide a better stability profile and may be suitable for administration via parenteral and oral routes.

Other aspects and advantages of the invention will be apparent from the following detailed description.

### DESCRIPTION OF THE INVENTION

Definitions:

The term Compound I, unless otherwise noted, refers to a compound having the following formula, 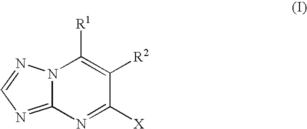
wherein:
R¹ is 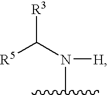
   or (C₆C₈) cycloalkyl optionally substituted with R⁸;
R² is a moiety of the group 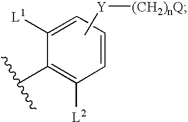
n is an integer of 2, 3, or 4;
X is F, Cl or Br;
Y is O, S, CH₂ or NR⁴;
Q is selected from —NR⁶R⁷ and —OH;
L¹ and L² are each independently H, F, Cl, Br, or CF₃;
R³is CF₃ or C₂F₅;
R⁴ and R⁵ are each independently H or (C₁-C₃) alkyl;
R⁶ and R⁷are each independently H or (C₁-C₃) alkyl; or R⁶ and R⁷may be optionally taken together with the nitrogen atom to which each is attached to form a 4 to 6 membered saturated heterocyclic ring containing 1-2 nitrogen atoms, 0-1 oxygen atoms or 0-1 sulfur atoms, and said 4 to 6 membered saturated heterocyclic ring may be optionally substituted with one or more R⁸; and
R⁸ is (C₁-C₃) alkyl.

The term Compound Ia refers to 5-chloro-6-{2,6-difluoro-4-[3-(methylamino)propoxy]phenyl}-N-[(1S)-2,2,2-trifluoro-1-methylethyl][1,2,4]triazolo[1,5-a]pyrimidin-7-amine and has the following structure:

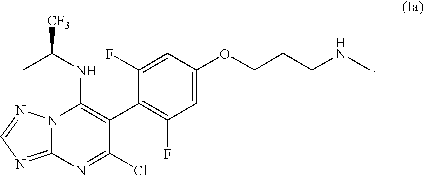

The term alkyl means a straight or branched chain alkyl moiety of 1 to 3 carbon atoms. A (C₁-C₃) alkyl includes methyl, ethyl, propyl, and isopropyl.

The term alkali metal hydroxide includes lithium, potassium or sodium hydroxide.

The term alkali metal carbonate includes lithium, potassium or sodium carbonate.

The term alkali metal hydride includes lithium, potassium or sodium hydride.

The term strong base means an alkali metal hydroxide, alkali metal carbonate and alkali metal hydride (e.g., sodium hydride).

Phenyl as used herein refers to a 6-membered carbon aromatic ring.

Cycloalkyl as used herein means a saturated carbocyclic monocyclic ring having from 6 to 8 carbon atoms optionally substituted with one or more (C₁-C₃) alkyl. Non-limiting represenative examples include: cyclohexyl, cycloheptyl and cyclooctyl.

As used herein a saturated heterocyclic ring is a 4 to 6 membered ring containing 1-2 nitrogen atoms, 0-1 oxygen atoms or 0-1 sulfur atoms and said ring may be optionally substituted with one or more (C₁-C₃) alkyl. Non-limiting representative examples include: morpholine, piperdine, pyrrolidine, piperazine, azetidine and N-methyl-piperazine.

The term “administer”, “administering”, or “administration”, as used herein refers to either directly administering a compound or pharmaceutically acceptable salt of the compound or a composition to an animal, or administering a prodrug derivative or analog of the compound or pharmaceutically acceptable salt of the compound or composition to the animal, which can form an equivalent amount of active compound within the animal's body.

The term “animal” as used herein includes, without limitation, a human, mouse, rat, guinea pig, dog, cat, horse, cow, pig, monkey, chimpanzee, baboon, or rhesus. In one embodiment, the animal is a mammal. In another embodiment, the animal is a human.

The term “effective amount” as used herein refers to an amount of a compound or pharmaceutically acceptable salt of a compound that, when administered to an animal, is effective to prevent, to at least partially ameliorate, or to cure, a condition from which the animal suffers or is suspected to suffer.

The term “carrier”, as used herein, shall encompass carriers, excipients, and diluents.

The term “pharmaceutically acceptable salt” as used herein refers to a salt of an acid and a basic nitrogen atom of a compound of the present invention. The term “pharmaceutically acceptable salt” may also include a hydrate of a compound or its pharmaceutically acceptable salt of the present invention. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, gentisinate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, palmitate, aspartate, phthalate, and pamoate. Preferred pharmaceutically acceptable salts of Compound Ia include succinate, acetate, mesylate, maleate, fumarate, tartarate, citrate, benzenesulphonate, L-aspartate, R-(−)-mandelate, sulphate, or palmitate; and each of the above mentioned salts may be anhydrous or a hydrate. Especially preferred pharmaceutically acceptable salt of Compound la is the succinate dihydrate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxy)amine; N-methyl-D-glucamine; morpholine; thiomorpholine; piperidine; pyrrolidine; and amino acids such as arginine, lysine, and the like.

The term “pharmaceutically acceptable acid” as used herein refers to any organic and inorganic acid that is acceptable for use in pharmaceutical applications from a toxicological perspective and does not adversely interact with the active ingredient. Exemplary acids include, but are not limited to, sulfuric, citric, cinnamic, acetic, oxalic, hydrochloric, hydrobromic, hydroiodic, nitric, phosphoric, isonicotinic, lactic, salicylic, tartaric, oleic, tannic, pantothenic, bitartaric, ascorbic, gentisinic, glycolic, gluconic, glucaronic, formic, benzoic, glutamic, pyruvic, methanesulfonic, ethanesulfonic, benzenesulfonic, p-toluenesulfonic, camphorsulfonic, napthalenesulfonic, propionic, aspartic, succinic, fumaric, maleic, malonic, mandelic, malic, palmitic, 1,2-benzenedicarboxylic acid, saccharic, pamoic, and similarly known acceptable acids. Preferred pharmaceutically acceptable acids include acetic acid, methanesulphonic acid, maleic acid, fumaric acid, tartaric acid, citric acid, benzenesulphonic acid, L-aspartic acid, R-(−)mandelic acid, sulphuric acid, or palmitic acid.

Further Illustration of the Invention:

The present invention provides pre-lyophilization compositions that provide freeze-dried compositions containing Compound I with improved potency retention and stability under storage conditions. Specifically, using the pre-lyophilization compositions of the invention, freeze-dried composition containing Compound Ia has been found to retain greater than 95% of initial potency after 176 days storage at 25° C. or at 40° C. The present invention also provides reconstituted compositions of Compound I or its pharmaceutically acceptable salt suitable for delivery parenterally or other routes of delivery.

The synthesis of Compound I (including Compound Ia) or its pharmaceutically acceptable salt is disclosed in US Publication No. 2005/0090508. This application disclosure of the compounds and their synthesis is hereby incorporated by reference herein.

A pre-lyophilization solution of Compound I or a pharmaceutically acceptable salt thereof such as the succinate dihydrate salt of Compound Ia, is formed by dissolving Compound I or its pharmaceutically acceptable salt in a suitable solvent selected from an organic solvent, an aqueous solvent or a mixture thereof. The solvent is sufficiently volatile to be removed under typical temperature and pressure conditions that are used in a commercial freeze-dryer. Additionally, the solubility of Compound I in the suitable solvent is sufficiently high to produce a material that is concentrated enough to permit practical applications of the drug. Typically, the concentration of Compound I or its pharmaceutically acceptable salt in the pre-lyophilized solutions ranges from about 1 mg/mL to about 100 mg/mL or up to the solubility limit, whichever is lower, preferably 2 mg/mL to 50 mg/mL, more preferably 5 mg/mL to 20 mg/mL, to provide a lyophilized form of Compound I or its pharmaceutically acceptable salt, which is suitable for preparing doses of Compound I of from about 1 to about 200 mg. Exemplary solvents include water, acetonitrile, ethanol, iso-propanol, t-butyl alcohol, DMSO, or a mixture thereof. The preferred solvent for dissolving the succinate dihydrate salt of Compound Ia comprises water.

These solvents or mixtures thereof are present in an amount of about 30% to about 49%, to about 50%, to about 60%, to about 70%, to about 80%, to about 90%, to about 95%, to about 99% Wt/Vol, although lower amounts of the individual solvents may be selected to provide a mixture to give a total solvent amount in the provided range.

In certain embodiments, the pre-lyophilization solution further contains bulking agents. These agents can be readily selected by one of skill in the art in view of the selected solvent or mixture thereof. Specifically, the solubility of typical water-soluble bulking agents such as sugars or polyols is reduced by the presence of organic solvents. In these embodiments, a mixture of organic solvent and water are used and the composition adjusted in order to balance an adequate concentration of drug with an effective concentration of added substance. Suitable bulking agents include carbohydrates such as mannitol, dextrose, dextran, or sucrose. Optionally, bulking agents such as polyvinylpyrrolidone, starch, lactose, trehalose or hydroxyethylstarch may be used in addition to carbohydrates mentioned hereinabove. Combinations of two or more of the bulking agents can also be used. Bulking agents can be used in a range of about 0.5% to about 10% Wt./Vol. in the pre-lyophilized solution, for example about 1%, about 2%, about 4%, about 6%, about 8% Wt./Vol.

In certain embodiments, the pre-lyophilization solution further contains a pharmaceutically acceptable acid for enhancing the stability of the lyophilized Compound I or Compound Ia of the invention. It has been found that the addition of a pharmaceutically acceptable acid can inhibit and/or minimize the formation of impurities, such as Dimer and Adduct as discussed above. Desirably, the lyphilized Compound I or Compound Ia of the invention retains greater than 95% potency for an extended period of time under a variety of storage conditions.

For example, it is advantageous to add a pharmaceutically acceptable acid to the pre-lyophilization solution to adjust its pH value to below about 8.5, such as about 7.0, about 6.5, about 6.0, about 5.5, about 5.0, about 4.5, about 4.0, about 3.5, about 3.0, about 2.5, about 2.0, about 1.5, or about 1.0. The pH value of the solution ranges preferably from about 2.0 to about 6.0, and more preferably from about 2.5 to about 4.0. This is the most preferred pH range for maximum stability of the succinate dihydrate salt of Compound Ia, where the formation of degradants (e.g., the Dimer and the acid Adduct) is minimized.

The pH of the solution can be adjusted using any suitable inorganic acid (e.g., HCl) or organic acid (e.g., acetic acid, methanesulphonic acid, maleic acid, fumaric acid, tartaric acid, citric acid, benzenesulphonic acid, L-aspartic acid, R-(−)mandelic acid, sulphuric acid, or palmitic acid), or base, as needed. Thereafter, the pre-lyophilization solution is subject to freeze-drying.

Freeze-drying can be performed using commercial freeze-dryers, such as are available from a variety of sources using manufacturer recommended settings. Desirably, the product is freeze-dried so that the lyophilized product contains less than about 2% wt/wt solvent or diluent. In one example, the product is loaded at about 20° C., frozen at about −35° C. to about −30° C.; held at or below about −30° C. for at least one hour, and followed by freezing the condenser and reducing the vacuum in the chamber to about 150 mTorr. The frozen solution is thermally treated by raising the shelf temperature to about 25° C., and holding for about 6 to about 19 hours, or until the product reaches 0° C. or higher. Alternatively, the frozen solution can be thermally treated by cycling the temperature from −40° C. to −5° C. and back to −20° C. Thereafter, the condenser can be started and the vacuum adjusted (e.g., to 100 mTorr) and the shelf temperature is raised to +10° C. Optionally, when the product temperature reaches +10° C., the product is subjected to secondary drying. Such secondary drying can begin when the shelf temperature has reached about 40° C. Secondary drying is performed under pressure, e.g., about 100 mTorr, overnight (e.g., about 12 to 18 hours), or for up to about 24 hours. Alternatively, this step may be performed for a shorter or longer time. Suitably, the freeze-drying results in a product having residual solvent in an amount of less than about 2% by weight of the final weight of solids in the lyophilized Compound I or its pharmaceutically acceptably salt. In addition or alternatively to the second step, other processing techniques can be used to further reduce the residual solvent in the resulting lyophilized material. Such processing techniques include nitrogen sweeps.

Advantageously, the lyophilized Compound I of the invention retains greater than 95% potency for an extended period of time under a variety of storage conditions. This lyophilized composition is suitable for preparing a variety of dosage forms for delivery to subject, and is particularly advantageous for formulation of liquid and oral dosage forms.

When preparing freeze-dried Compound I or its pharmaceutically acceptable salt for reconstitution, a suitable solvent is selected. An effective solvent for reconstitution is biocompatible, dissolves adequate quantities of drug in relatively small volumes and prevents precipitation of the drug during injection into body fluids or dilution in intravenous infusion solutions. In one embodiment, parenterally acceptable amphiphilic compounds are combined with water, organic solvents or a mixture thereof. Examples of suitable amphiphilic compounds includes polysorbate 20, 60 or 80, ethoxylated oils, such as PEG-35 castor oil (e.g., Cremophor EL), fatty acid-PEG esters, such as Solutol HS, vitamin E tocopherol propylene glycol succinate (Vitamin E TPGS), sucrose-fatty acid esters, bile salts, phospholipids and combinations of bile salts with phospholipids. The concentration of amphiphilic can range from 2% to 100% w/v in the reconstitution solvent. Alternatively, in certain embodiments, the amphiphile can be incorporated with Compound I or its pharmaceutically acceptable salt in the pre-lyophilization formulation. In such embodiments, reconstitution can be accomplished using either water or a combination of water and organic solvent.

When Compound I or its pharmaceutically acceptable salt is reconstituted according to this invention, the reconstituted formulation can contain concentrations of Compound I from about 0.05 mg/mL, from about 2.5 mg/mL, from about 5 mg/mL or from about 10 mg/mL up to approximately 50 mg/mL. The concentrate can be mixed with the diluent up to approximately 1 part concentrate to 1 part diluent, to give compositions having concentrations of Compound I from about 1 mg/mL, from about 5 mg/mL, from about 10 mg/mL, from about 20 mg/mL, up to approximately about 25 mg/mL. This invention also covers compositions having lesser concentrations of Compound I in the co-solvent concentrate, and compositions in which one part of the concentrate is mixed with greater than 1 part of the diluent, e.g., concentrate: diluent in a ratio of about 1:1.5, 1:2, 1:3, 1:4 or 1:5 v/v, and so on, to Compound I compositions having a Compound I concentration down to the lowest levels of detection. A suitable diluent can readily be selected by one of skill in the art, in view of the route of delivery. For example, the diluent can be aqueous, primarily aqueous, e.g., glucose solution, saline, buffered saline, 0.9% sodium chloride injection, 5% dextrose injection, lactated ringers injection, or non-aqueous.

The reconstituted compositions of this invention can be used to produce a parenteral dosage form. Such a dosage form may be suitable for administration by either direct injection or by addition to sterile infusion fluids for intravenous infusion.

The compositions of the invention may be produced in the form of a kit of parts. Such a kit is suitable for preparing an aqueous pharmaceutical composition. Typically, the kit will contain at least a first container having the lyophilized Compound I or its pharmaceutically acceptable salt composition of the invention and optionally a second container having a physiologically acceptable solvent therefore. Other components may include vials, stirrers, lids, instructions for reconstitution, mixing, storage and/or, use. Optionally, other active ingredients to be administered in a regimen with the lyophilized or reconstituted Compound I or its pharmaceutically acceptable salt may also be provided. The invention also includes a pharmaceutical pack containing a course of treatment for one individual mammal, wherein the pack contains Compound I or its pharmaceutically acceptable salt and one or more of the kit components described above.

The following examples are illustrative of the present invention. The present invention is not limited to the percentages, components and techniques described herein.

### EXAMPLES

Examples 1 to 4 provide illustrative lyophilized compositions of the present invention.

### Example 1

A 5 mg strength vial was lyophilized from a 2 mg/mL bulk solution using the dihydrate succinate salt of Compound Ia. Since the concentration of the active ingredient alone was not adequate to produce a strong lyophile cake, mannitol at 40 mg/mL was utilized as a bulking agent and the bulk solution pH was about 4.9. The lyophile possessed good physical characteristics. Upon reconstitution with 2.46 mL of water to 2 mg/mL, the pH was about 4.9, the same as the bulk solution pH before lyophilization. The reconstituted solution was stored at room temperature, assayed at time=0, 18, 24, 42, and 66 hours and shown to be stable for at least 66 hours with no loss in strength and no degradants, indicating a 3-day use period after reconstitution. However, stressed stability study of the lyophile vials shows that after 10 weeks at 40° C., both the dimer (5.7%) and the succinic acid adduct (2.5%) were formed.

### Example 2

A 100 mg strength vial was prepared by lyophilizing a 20 mg/mL aqueous solution of the succinate dihydrate salt of Compound Ia with 8% Wt/Vol mannitol, pH adjusted to about 3.1 using an appropriate amount of hydrochloric acid. The fill volume was 5.25 mL per vial (for a 5% overage) using a 10-mL vial with 20-mm stopper. The freeze-dried material was found to retain greater than 95% initial potency after 76 days storage at 25° C. and after 140 days storage at 40° C.

### Example 3

The pre-lyophilized solution was 20 mg/mL Compound Ia, 0.4 mg/mL Adduct, and 3.4% mannitol. The mannitol amount was selected to provide a nearly isotonic solution. The pH of the bulk solution was adjusted to about 3 with hydrochloric acid. Fill volume per vial was 5.3 mL to give a 6% overage to the label claim of 100 mg Compound Ia and 2 mg of Adduct. The amount of components per vial and the total batch quantities are summarized in Table 1.

TABLE 1
Formulation Composition Per Vial

| Component | Amount Per Vial^{(b)} | | Batch Quantity | |
|---|---|---|---|---|
| Compound Ia @ 100%^{(a)} | 0.106 | g | 39.08 | g |
| Adduct @ 100%^{(a)} | 0.00212 | g | 0.60 | g |
| Mannitol | 0.1802 | g | 49.56 | g |
| Hydrochloric Acid, 37.7% NF | 0.02053 | g | | |
| Water for Injection, USP^{(c)} | q.s. 5.3 | mL^{(b)} | 5.65 | g |
| | or 5.3663 | g | 1381.34 | g |
| Total | 5.3663 | g | 1476.23 | g |

^{(a)}If potency of the drug is less than 100%, the input must be adjusted to give claimed potency.
^{(b)}Based on a 5.3 mL fill into a 10 mL flint vial.

Each lyophile vial is reconstituted with 5.1 mL of WFI (water for injection) to give a deliverable volume of 5 mL at 20 mg/mL of Compound Ia and 0.4 mg/mL of Adduct.

The lyophilization process is as follows:
A. Load filled trays onto freeze dryer shelves. Insert thermocouples into vials, continue to cool lyophilizer shelves to −35° C.;
B. Allow product temperature to reach −30° C.;
C. Hold product at temperatures <=−30° C. for at least 1 hour;
D. Freeze condenser;
E. Pull vacuum in chamber to 150 mTorr;
F. Ramp shelf temperature to +25° C. in one hour. Hold at this temperature for 19 hours, or until product reaches 0° C. or higher;
G. Ramp shelf temperature to +40° C. in one hour and hold at this temperature for 12 hours;
H. Ramp shelf temperature to 25° C. in one hour; and
I. Break vacuum with nitrogen, stopper vials.

The freeze-dried material was found to retain greater than 95% initial potency after 6 months at 25° C./60% RH (relative humidity) and after 3 months at 40° C./75% RH.

### Example 4

The formulation strength of 20 mg/vial was prepared from a 10 mg/mL Compound Ia solution with 4% mannitol and 0.2% hydrochloric acid, NF for pH adjustment (the pH of the resulting solution was about 3.0). The fill volume is 2.12 mL per vial to give a 6% overage. After filtration, the solution is filled into 5 mL flint vials for lyophilization. The composition and unit input are shown as in Table 2.

TABLE 2
Composition of Compound Ia for IV Injection (20 mg/vial)

| Ingredient | % Wt/Vol | Input/Vial |
|---|---|---|
| Compound Ia @ 100% ^{a} | 1% | 0.0212 g |
| Mannitol, USP, Pyrogen Free | 4% | 0.0848 g |
| Hydrochloric Acid 36.5-38%, NF | 0.2% | 0.0042 g |
| Water for Injection, USP ^{b} | q.s. to 100% | 2.045 g |
| Total | 100% | 2.1552 g (2.12 mL) |

^{a} If potency of drug substance is less than 100%, the input must be adjusted to the claimed potency.
^{b} Water is removed during lyophilization process.

The lyophilization process is as follows:
1. Weigh the active ingredient into a suitable container;
2. Add mannitol to the container in step #1;
3. Add 80% required WFI at 35-45° C. to the container in step #2;
4. To the container in step #3, add HCl;
5. Qs to final weight with WFI;
6. Mix until a solution is formed;
7. Allow solution to cool to 25° C.±5° C., check weight, q.s. if necessary;
8. Take and record the pH;
9. Take a bioburden sample;
10. Pre-filter through a 0.45 μ filter;
11. Aseptically filter it through a 0.2 Φ sterile filter;
12. Fill 2.12 mL into each pre-sterilized 5 mL vial and half-insert one lyophilization stopper;
13. Take an in-process potency sample;
14. Begin the lyophilization procedure;
   A. Load filled trays onto lyophilizer shelves at 20° C. Insert thermocouples into vials, cool lyophilizer shelves to −35° C. or lower;
   B. Allow product temperature to reach −30° C. over 240 min.;
   C. Hold product at temperatures <=−30° C. for at least one hour;
   D. Freeze condenser to −50° C.;
   E. Pull vacuum in chamber to 200 μBar;
   F. Ramp shelf temperature to +25° C. in one hour and hold at this temperature until product reaches 15° C. Hold at 15° C. for one hour;
   G. Ramp shelf temperature to +40° C. in one hour and hold at this temperature for 16 hours;
   H. Ramp shelf temperature to 25° C. in one hour; and
   I. Break vacuum with nitrogen to about 500 mBar, stopper vials; and
15. Crimp seal vial with aluminum caps.

Each lyophilized vial is to be reconstituted with 5.2 mL of sterile water to yield a volume of 5.3 mL of which 5.0 mL can be withdrawn for injection or further dilution in IV admixtures for infusion. The freeze-dried material was found to retain greater than 95% initial potency after 18 months at 25° C./60% RH and after 6 months at 40° C./75% RH.

We claim:

1. A composition useful for preparing freeze-dried Compound I, or a hydrate thereof, or a pharmaceutically acceptable salt of Compound I or hydrate thereof, said composition comprising:
(a) Compound I or a hydrate thereof, or a pharmaceutically acceptable salt of Compound I or hydrate thereof, having the following structure: 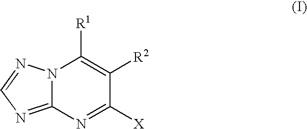
wherein:
R¹ is 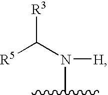
or (C₆C₈) cycloalkyl optionally substituted with R⁸;
R² is a moiety of the group 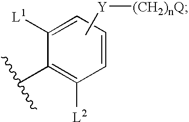
n is an integer of 2, 3, or 4;
X is F, Cl or Br;
Y is O, S, CH₂ or NR⁴;
Q is selected from —NR⁶R⁷ and —OH;
L¹ and L² are each independently H, F, Cl, Br, or CF₃;
R³ is CF₃ or C₂F₅;
R⁴ and R⁵ are each independently H or (C₁-C₃) alkyl;
R⁶ and R⁷ are each independently H or (C₁-C₃) alkyl; or R⁶ and R⁷ may be optionally taken together with the nitrogen atom to which each is attached to form a 4 to 6 membered saturated heterocyclic ring containing 1-2 nitrogen atoms, 0-1 oxygen atoms or 0-1 sulfur atoms, and said 4 to 6 membered saturated heterocyclic ring may be optionally substituted with one or more R⁸; and
R⁸ is (C₁-C₃) alkyl;
(b) a bulking agent;
(c) an effective amount of a pharmaceutically acceptable acid for enhancing the stability of said freeze-dried Compound I or a hydrate thereof, or a pharmaceutically acceptable salt of Compound I or hydrate thereof; and
(d) a solvent.

2. A composition according to claim 1, wherein said Compound I is Compound Ia having the following structure:

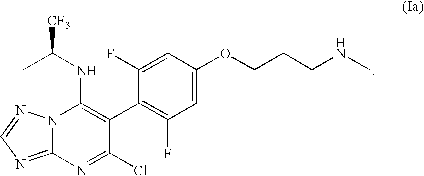

3. A composition according to claim 1, wherein said composition has a pH in a range of about 2.0 to about 6.0.

4. A composition according to claim 1, wherein said composition has a pH in a range of about 2.5 to about 5.0.

5. A composition according to claim 1, wherein said composition has a pH in a range of about 2.7 to about 4.0.

6. A composition according to claim 2, wherein said composition has a pH in a range of about 2.7 to about 4.0.

7. A composition according to claim 1, wherein said composition comprises about 1 mg/mL to about 100 mg/mL of said Compound I or a pharmaceutically acceptable salt thereof.

8. A composition according to claim 1, wherein said composition comprises about 1% to about 10% wt/vol of said bulking agent.

9. A composition according to claim 8, wherein said bulking agent is a carbohydrate.

10. A composition according to claim 9, wherein said carbohydrate is mannitol, dextrose, dextran, or sucrose.

11. A composition according to claim 9, wherein said carbohydrate is mannitol.

12. A composition according to claim 11, wherein said Compound I is Compound Ia, and said pharmaceutically acceptable salt is succinate dihydrate.

13. A composition according to claim 2, wherein said pharmaceutically acceptable acid in (c) is hydrochloric acid, acetic acid, methanesulphonic acid, maleic acid, fumaric acid, tartaric acid, citric acid, benzenesulphonic acid, L-aspartic acid, R-(−)mandelic acid, sulphuric acid, or palmitic acid.

14. A composition according to claim 2, wherein said pharmaceutically acceptable salt in (a) is succinate, acetate, mesylate, maleate, fumarate, tartarate, citrate, benzenesulphonate, L-aspartate, R-(−)-mandelate, sulphate, or palmitate.

15. A composition according to claim 2, wherein said pharmaceutically acceptable salt in (a) is succinate, fumarate, or R-(−)-mandelate.

16. A composition according to claim 2, wherein said pharmaceutically acceptable salt in (a) is succinate dihydrate, and wherein said pharmaceutically acceptable acid in (c) is hydrochloric acid.

17. A method for preparing a lyophilized formulation of Compound I, or a hydrate thereof, or a pharmaceutically acceptable salt of Compound I or hydrate thereof, said method comprising the step of freeze-drying a composition according to claim 1.

18. A method for preparing a lyophilized composition of Compound Ia, or a hydrate thereof, or a pharmaceutically acceptable salt of Compound la or hydrate thereof, said method comprising the step of:
(a) preparing a pharmaceutical composition having a pH in the range of about 2.0 to about 5.0 and comprising about 1 mg/mL to about 50 mg/mL of Compound Ia or a hydrate thereof, or a pharmaceutically acceptable salt of Compound Ia or hydrate thereof, about 1% to about 10% mannitol, and water; and
(b) freeze-drying said composition to form said lyophilized composition.

19. A lyophilized composition of Compound I, or a hydrate thereof, or a pharmaceutically acceptable salt of Compound I or hydrate thereof, formed by freeze-drying a solution according to claim 1.

20. A lyophilized composition according to claim 19, wherein said Compound I is Compound Ia, and said pharmaceutically acceptable salt is succinate dihydrate.

21. A method for preparing Compound I, or a hydrate thereof, or a pharmaceutically acceptable salt of Compound I or hydrate thereof, for delivery in liquid form, said method comprising the step of reconstituting a lyophilized composition of Compound I or its pharmaceutically acceptable salt according to claim 19 with a parenterally acceptable solvent to form a liquid dosage form of Compound I or its pharmaceutically acceptable salt.

22. The method according to claim 21, wherein said parenterally acceptable solvent comprises water.

23. The method according to claim 21, wherein said Compound I is Compound Ia, and said pharmaceutically acceptable salt is succinate dihydrate.

24. A liquid dosage form of Compound I, or a hydrate thereof, or a pharmaceutically acceptable salt of Compound I or hydrate thereof, formed according to the method of claim 21.

25. A liquid dosage form according to claim 24, wherein said Compound I is Compound Ia, and said pharmaceutically acceptable salt is succinate dihydrate.

26. A method of enhancing storage stability of Compound I, or a hydrate thereof, or a pharmaceutically acceptable salt of Compound I or hydrate thereof, said method comprising the step of lyophilizing a composition having a pH in the range of about 2.0 to about 5.0 and comprising about 1 mg/mL to about 50 mg/mL of Compound I, or a hydrate thereof, or a pharmaceutically acceptable salt of Compound I or hydrate thereof, about 1% to about 10% mannitol, and water.

27. The method according to claim 26, wherein said Compound I is Compound Ia, and said pharmaceutically acceptable salt is succinate dihydrate.

28. A kit comprising a container for the lyophilized Compound I, or a hydrate thereof, or a pharmaceutically acceptable salt of Compound I or hydrate thereof, according to claim 19.

29. A kit comprising a container for the lyophilized Compound Ia, or a hydrate thereof, or a pharmaceutically acceptable salt of Compound I or hydrate thereof, according to claim 20.

30. A kit according to claim 27, further comprising a parenterally acceptable solvent for reconstitution thereof.

31. A method of treating or inhibiting the growth of cancerous tumor cells and associated diseases in a mammal in need thereof by administering an effective amount of a liquid dosage form of Compound I, or a hydrate thereof, or a pharmaceutically acceptable salt of Compound I or hydrate thereof, as defined in claim 24.

32. A method of promoting tubulin polymerization in a tubulin containing system which comprises contacting said tubulin containing system with an effective amount of a liquid dosage form of Compound I, or a hydrate thereof, or a pharmaceutically acceptable salt of Compound I or hydrate thereof, as defined in claim 24.

33. A method of stabilizing microtubules in a tubulin containing system which comprises contacting said tubulin containing system with an effective amount of a liquid dosage form of Compound I, or a hydrate thereof, or a pharmaceutically acceptable salt of Compound I or hydrate thereof, as defined in claim 24.

34. A method for the treatment or prevention of tumors that express multiple drug resistance (MDR) or are resistant because of MDR in a mammal in need thereof, which method comprises administering to said mammal an effective amount of a liquid dosage form of Compound I, or a hydrate thereof, or a pharmaceutically acceptable salt of Compound I or hydrate thereof, as defined in claim 24.

35. A method of treating or inhibiting the growth of cancerous tumor cells and associated diseases in a mammal in need thereof by administering an effective amount of a liquid dosage form of Compound I, or a hydrate thereof, or a pharmaceutically acceptable salt of Compound I or hydrate thereof, as defined in claim 24.

36. A method of promoting tubulin polymerization in a tubulin containing system which comprises contacting said tubulin containing system with an effective amount of a liquid dosage form of Compound Ia, or a hydrate thereof, or a pharmaceutically acceptable salt of Compound I or hydrate thereof, as defined in claim 25.

37. A method of stabilizing microtubules in a tubulin containing system which comprises contacting said tubulin containing system with an effective amount of a compound of a liquid dosage form of Compound Ia, or a hydrate thereof, or a pharmaceutically acceptable salt of Compound I or hydrate thereof, as defined in claim 25.

38. A method for the treatment or prevention of tumors that express multiple drug resistance (MDR) or are resistant because of MDR in a mammal in need thereof, which method comprises administering to said mammal an effective amount of a liquid dosage form of Compound Ia, or a hydrate thereof, or a pharmaceutically acceptable salt of Compound I or hydrate thereof, as defined in claim 25.

39. A method of treating or inhibiting the growth of cancerous tumor cells and associated diseases in a mammal in need thereof by administering an effective amount of a liquid dosage form of Compound Ia, or a hydrate thereof, or a pharmaceutically acceptable salt of Compound I or hydrate thereof, as defined in claim 25.
